SEQUENCE LISTING (20 sequences)

SEQ ID NO: 1 - DNA, 205 bp, Homo sapiens
tttaaaaacctccctatcacctccgatcactgttgaaaaagcattaaactgtaagaaggggttagtattgggggaagcatgtcgtttctaaggatgggaaaggaaaatgaagtgcttctcctccctgatccaagagaggcagcttcatgaaacttctgtatgaaaatgggagcgtctgtaggaagagggactctatttacataac

SEQ ID NO: 2 - DNA, 20 bp, Artificial Sequence
  Downstream primer R1
ggggaagcatgtcgtttcta

SEQ ID NO: 3 - DNA, 20 bp, Artificial Sequence
  Upstream primer F1
tcttggatcagggaggagaa

SEQ ID NO: 4 - DNA, 20 bp, Artificial Sequence
  Downstream primer R2
acctccctatcacctccgat

SEQ ID NO: 5 - DNA, 20 bp, Artificial Sequence
  Upstream primer F2
cttcctacagacgctcccat

SEQ ID NO: 6 - DNA, 20 bp, Artificial Sequence
  Downstream primer R3
gggttagtattgggggaagc

SEQ ID NO: 7 - DNA, 20 bp, Artificial Sequence
  Upstream primer F3
atgaagctgcctctcttgga

SEQ ID NO: 8 - DNA, 21 bp, Artificial Sequence
  Downstream primer R4
ggatgggaaaggaaaatgaag

SEQ ID NO: 9 - DNA, 20 bp, Artificial Sequence
  Upstream primer F4
ttcctacagacgctcccatt

SEQ ID NO: 10 - DNA, 25 bp, Artificial Sequence
  Probe1
cacttcattttcctttcccatcctt

SEQ ID NO: 11 - DNA, 24 bp, Artificial Sequence
  Probe2
cctccctgatccaagagaggcagc

SEQ ID NO: 12 - DNA, 23 bp, Artificial Sequence
  Upstream primer SRGAP2-F
cgatactcaggtcaaaggtaagg

SEQ ID NO: 13 - DNA, 22 bp, Artificial Sequence
  Downstream primer SRGAP2-R
ctgcaaatcacggtggaaatac

SEQ ID NO: 14 - DNA, 24 bp, Artificial Sequence
  SRGAP2-Probe
tgcaaatgctctgtggactggtga

SEQ ID NO: 15 - DNA, 20 bp, Artificial Sequence
  Upstream primer Qhomo2-F
gtgggtgggaagagggaagc

SEQ ID NO: 16 - DNA, 24 bp, Artificial Sequence
  Downstream primer Qhomo2-R
actcggcattcacacatttctcag

SEQ ID NO: 17 - DNA, 23 bp, Artificial Sequence
  Qhomo2-Probe
cagcagtggcgtgtgggaacctg

SEQ ID NO: 18 - DNA, 22 bp, Artificial Sequence
  Upstream primer Alu-F
gtcaggagatcgagaccatcct

SEQ ID NO: 19 - DNA, 17 bp, Artificial Sequence
  Downstream primer Alu -R
agtggcgcaatctcggc

SEQ ID NO: 20 - DNA, 25 bp, Artificial Sequence
  Alu -Probe
agctactcgggaggctgaggcagga

(19) United States
(12) Patent Application Publication
(10) Pub. No.: US 2023/0279504 A1
(43) Pub. Date: Sep. 7, 2023

(54) A fluorescent quantitative PCR technology-based method for distinguishing human DNA

(71) Applicant: EYECURE THERAPEUTICS, INC. (JIANGSU), Wuxi (CN)

(72) Inventors: Li FENG, Wuxi (CN); Xiaoyan LENG, Wuxi (CN); Yunjuan WANG, Wuxi (CN); Hui LIU, Wuxi (CN)

(21) Appl. No.: 17/790,490

(22) PCT Filed: Jan. 25, 2019
(86) PCT No.: PCT/CN2021/073569

§ 371 Date: Jun. 30, 2022

(30) Foreign Application Priority Data
Dec. 30, 2019 (CN) 201911394885.7

(51) Int. Cl. C12Q 1/6888

(52) U.S. Cl. CPC C12Q 1/6888; C12Q 2600/158

(57) ABSTRACT

The invention discloses a fluorescent quantitative PCR technology-based method for distinguishing human DNA, and a fluorescent quantitative PCR technology-based composition or kit for distinguishing human DNA, comprising primers and/or probes of nucleotide sequences.

### TECHNICAL FIELD

The invention relates to a fluorescent quantitative PCR technology-based method for distinguishing human DNA, and detecting primers and probes.

### TECHNICAL BACKGROUND

In recent years, with the rapid development of cell drugs, more and more cells are being developed into cell therapy drugs, gradually progressing from research to clinic.

Cell therapy may be the trend of novel drug research and development. An indispensable step in determining the curative and side effects of cell therapy drugs is to study its pharmacokinetics in vivo. Unlike traditional chemical drugs, with regard to the dynamic change regularity of cell drugs after entering the body, including the process and characteristics of their absorption, distribution, metabolism and excretion, so far there is no globally established method to study the in vivo pharmacokinetics of cell drugs.

Nonclinical pharmacokinetic studies, especially animal experiments, play an important role in the evaluation of novel drug research and development. By studying their dynamics in vivo and predicting the appropriate dosage in treatment, it is of great help to reduce the uncertainty of cell therapy, increase curative effect and minimize side effects. To take the results of animal experiments as a method to evaluate the therapeutic effect of cell products and connect them with clinical effects, it is inevitably needed to find a highly sensitive and fully validated method to quantify the distribution of cell products in animal models. Compared to experimental rat and rabbit, experimental monkey is a very important kind of experimental animal in the field of novel drug research. Because of its high similarity with human, experimental monkey is considered to be an essential tool in drug evaluation. Before applying cell drugs to human body, it is necessary to carry out systematic evaluation in animals having a close genetic relationship with human beings.

At present, the methods to study the distribution of cells in experimental monkeys mainly include in vivo imaging, fluorescent protein labeling, immunohistochemistry, qPCR and the like, each of which has its advantages and disadvantages. For example, Magnetic Resonance Imaging (MRI) reflects the distribution of cells in vivo. This method needs to label cells in vitro and detects the cells injected into the body by imaging, so as to decide survival and clearance of the cells, and has high sensitivity and long labeling duration. However, some of the labeling methods may have an impact on cell activity. It is reported that after MRI labeling of bone marrow mesenchymal stem cells, the ability of cell differentiation is impaired. Moreover, the MRI sensitivity is relatively insufficient because iron oxide particles (MRI markers) released after cell death give rise to nonspecific development and so as false positive result. For example, green fluorescent protein marker (GFP), this method enables cells to express fluorescent protein by genetic modification. The green fluorescent signal can be observed directly under fluorescent microscope, which render the method an advantage of easy detection. However, as the genetic material of the cell has been changed, there is no guarantee that such a change could be 100% stable. Any cell labeling method can potentially change the cell, and influence progeny cell differentiation or the like. The immunohistochemistry method does not require cell labeling, but needs a great quantity of sectioning and microscopic observation. Meanwhile, due to the similarity between human and monkey, there is antigen cross between human and monkey. Moreover, this method is difficult to standardize and only semi-quantitative results can be achieved.

qPCR method is a very sensitive and relatively simple-to-operate method, and it is expected to achieve quantitative analysis. To achieve quantitative analysis of human-derived cells in lab monkeys by detecting DNA in blood and tissue samples with the qPCR method, it is first necessary to find sequences and primers that can distinguish human DNA from animal model DNA. Although some differences in the expression of certain specific genes between humans and monkeys are noted in the literature, there are currently false negatives for genes that distinguish human and monkey cells because after the experimental cells are injected into monkeys, the cells may differentiate into cells that no longer express that specific gene. That is, human cells are present in monkey tissues, but their presence cannot be detected. For example, primers designed based on a human specific genetic fragment -Alu gene as a molecular marker were hoped to achieve the distinction between human DNA and monkey DNA by qPCR, and eventually to achieve the detection of human cells in monkey cells or tissues. Unfortunately, these primers can only distinguish human from rodent.

For example, Pengyue Song et al. reported in 2012 that, an efficient and repeatable PCR method based on DNA specific primers can detect xenografted human cells in mouse tissue. In 2015, Julie et al. published an article which reported a method to measure by qPCR the number of human cells transplanted in rats and mice. However, few articles have reported that human and monkey DNA can be distinguished by qPCR.

### SUMMARY OF THE INVENTION

In one aspect, the invention discloses a DNA sequence, wherein the DNA sequence is selected from the group consisting of SEQ ID NO:1 or a fragment thereof, and the reverse complement sequence of SEQ ID NO:1 or a fragment thereof, wherein the DNA sequence is used to distinguish between human DNA and non-human animal DNA in samples mixed with human and non-human animal tissues.

Sequence of SEQ ID NO:1:

tttaaaaacctccctatcacctccgatcactgttgaaaaagcattaaactgtaagaaggggttagtattgggggaagcatgtcgtt tctaaggatgggaaaggaaaatgaagtgcttctcctccctgatccaagagaggcagcttcatgaaacttctgtatgaaaatgggagcgt ctgtaggaagagggactctatttacataac

In another aspect, the invention discloses use of a DNA sequence in preparation of reagent or kit for distinguishing between human DNA and non-human animal DNA in samples mixed with human and non-human animal tissues, wherein the DNA sequence is selected from the group consisting of SEQ ID NO:1 or a fragment thereof, and the reverse complement sequence of SEQ ID NO:1 or a fragment thereof.

The invention further discloses use of a reagent for detecting DNA sequence in preparation of reagent or kit for distinguishing between human DNA and non-human animal DNA in samples mixed with human and non-human animal tissues, wherein the DNA sequence is selected from the group consisting of SEQ ID NO:1 or a fragment thereof, and the reverse complement sequence of SEQ ID NO:1 or a fragment thereof.

In one specific embodiment, the DNA sequence is SEQ ID NO:1, or the reverse complement sequence thereof, or the partial fragment of the full length sequences, wherein the fragment is SEQ ID NO:1 or the reverse complement sequence thereof, lacking 1-70 nucleotides at the 5′ and/or 3′ terminal, wherein the fragment sequence can still be used to distinguish between human DNA and non-human animal DNA in samples mixed with human and non-human animal tissues.

In one specific embodiment, the reagent for detecting DNA sequence is selected from primers and probes required for amplifying the DNA sequence through PCR technology.

In one specific embodiment, the probe sequence is shown in SEQ ID NO:10.

In one specific embodiment, the probe is provided with a detection marker, and the detection marker is preferably selected from the group consisting of FAM, TET, Alexa 488, Alexa 532, CF, HEX, VIC, ROX, Texas Red, QuasarFITC, cy3, cy5, 6-joe, EDANS, rhodamine 6G, TMR, TMRITC, x-rhodamine, Texas red, biotin and avidin.

In one specific embodiment, the primer sequence is selected from the group consisting of SEQ ID NO:2 and SEQ ID NO:3; SEQ ID NO:2 and SEQ ID NO:5; SEQ ID NO:4 and SEQ ID NO:3; SEQ ID NO:6 and SEQ ID NO:7; and SEQ ID NO:4 and SEQ ID NO:7.

In one specific embodiment, the non-human animal is selected from the group consisting of rhesus monkey, green monkey, cynomolgus monkey, rat, mouse and rabbit.

In one specific embodiment, the mixed human and non-human animal tissue is tissue or blood sample of a non-human animal such as rhesus monkey mixed with human DNA; the human DNA is derived from human cells. In one specific embodiment, the human DNA is derived from DNA in human retinal pigment epithelial cells.

In another aspect, the invention discloses a composition comprising primers and probes, wherein sequence of the probe is shown in SEQ ID NO:10, the primer sequence is selected from the group consisting of SEQ ID NO:2 and SEQ ID NO:3; SEQ ID NO:2 and SEQ ID NO:5; SEQ ID NO:4 and SEQ ID NO:3; SEQ ID NO:6 and SEQ ID NO:7; and SEQ ID NO:4 and SEQ ID NO:7.

Wherein, the probe is provided with a detection marker, and the detection marker is preferably selected from group consisting of FAM, TET, Alexa 488, Alexa 532, CF, HEX, VIC, ROX, Texas Red, QuasarFITC, cy3, cy5, 6-joe, EDANS, rhodamine 6G, TMR, TMRITC, x-rhodamine, Texas red, biotin and avidin.

In another aspect, the invention also discloses a kit comprising the aforementioned composition.

Furthermore, the invention also discloses a method for distinguishing between human DNA and non-human animal DNA sequences in mixed human and non-human animal tissue without diagnostic or therapeutic purpose, wherein PCR amplification of DNA is performed on a sample mixed with human and non-human animal tissue using the aforementioned composition or the aforementioned kit.

One specific embodiment comprises the following steps:
1) performing DNA extraction of a sample mixed with human and non-human animal tissue using the composition and cellular DNA extraction kit;
2) performing Taqman qPCR amplification using the primers and probes in the aforementioned composition or aforementioned kit;
3) collecting fluorescent signal, calculating cycle threshold (CT) value, calculating the concentration of human DNA in the samples.

Wherein, the non-human animal is selected from the group consisting of rhesus monkey, green monkey, cynomolgus monkey, rat, mouse and rabbit.

Wherein, the mixed human and non-human animal tissue is tissue or blood sample of a nonhuman animal such as rhesus monkey mixed with human DNA; the human DNA is derived from human cells, preferably from retinal pigment epithelial cells.

### BENEFICIAL EFFECTS

The invention has found a segment of DNA sequence in chromosome of human genome; the said DNA sequence is human specific. Some primers and probes are designed based on the said DNA sequence and they can distinguish DNA from human and many other species. In practical application, the invention can achieve the detection of human specific DNA from experimental animal DNA.

### BRIEF DESCRIPTION OF THE DRAWINGS

FIG. 1 shows the amplification curve for detecting DNA from different species using R1, F1 and Probe1, targeting the DNA sequence shown in SEQ ID NO:1 in the invention. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 2 shows the amplification curve for detecting DNA from different species using R2, F2 and Probe1, targeting the DNA sequence shown in SEQ ID NO:1 in the invention. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 3 shows the amplification curve for detecting DNA from different species using R2, F1 and Probe1, targeting the DNA sequence shown in SEQ ID NO:1 in the invention. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 4 shows the amplification curve for detecting DNA from different species using R3, F3 and Probe1, targeting the DNA sequence shown in SEQ ID NO:1 in the invention. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 5 shows the amplification curve for detecting DNA from different species using R2, F3 and Probe1, targeting the DNA sequence shown in SEQ ID NO:1 in the invention. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 6 shows the amplification curve for detecting DNA from different species, targeting the SRGAP2 gene. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 7 shows the amplification curve for detecting DNA from different species, targeting the Qhomo2 gene. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 8 shows the amplification curve for detecting DNA from different species, targeting the Alu gene. (\textcircled{1} human retinal pigment epithelial cell DNA; \textcircled{2} rhesus monkey DNA; \textcircled{3} green monkey DNA; \textcircled{4} cynomolgus monkey DNA; \textcircled{5} rat DNA; \textcircled{6} mouse DNA; \textcircled{7} rabbit DNA).

FIG. 9 shows the typical standard curve for detecting DNA sequence from human retinal pigment epithelial cell by real-time fluorescent quantitative PCR.

### DETAILED DESCRIPTION

Detailed explanations are made as below to further describe the invention.

Unless defined otherwise, all technical and scientific terms used herein have the same meaning as commonly understood by one of ordinary skill in the art to which the invention pertains.

In this invention, the singular forms “a,” and “the” include plural reference, unless the context clearly dictates otherwise.

As used herein, the term “non-human animal” includes all vertebrates, such as mammals and non-mammals, such as non-human primates, sheep, canines, felines, equines, bovines, chickens, rats, mice, amphibians, reptiles, and the like. In specific examples, non-human animals are selected from the group consisting of rhesus monkey, green monkey, cynomolgus monkey, rat, mouse and rabbit.

As described herein, the term “DNA sequence” in the invention refers to a DNA sequence encoding protein, such as, but not limited to, a DNA sequence existing in cell genome and encoding protein.

As described herein, the term “probe” in the invention refers to an oligonucleotide molecule provided with detection marker. The term “detection marker” in the invention refers to a molecule or group that generates detection signal. Detection marker includes, but not limited to, fluorophores (for example, see European Patent EP144914), radioisotopes (for example, see U.S. Pat. Nos. 4,358,535 and 4,446,237), antibodies, enzymes and oligonucleotides (for example, oligonucleotide barcodes).

Examples of fluorophores include, but not limited to, 6-carboxyfluorescein (FAM), Tetrachlorofluorescein (TET), Alexa (for example, Alexa 488, Alexa 532), CF, HEX, VIC, ROX, Texas Red, QuasarFITC, cy3, cy5, 6-joe, EDANS, rhodamine 6G (P6G) and derivatives thereof (tetramethyirhodamine (TMR), tetramethylrhodamine isothiocyanate (TMRITC), x-rhodamine, Texas red, probes with trade names of “BODJPY FL”, “BODJPY FL/C3”, “BODIPY EL/C6”, “BODIPY 5-FAM”, “BODJPY TMR”, “BODJPY TR”, “BODJPY R6G”, “BODJPY 564” and “BODJPY 581” produced by Molecular Probes, Inc., located in Eugene, Oreg., USA, and derivatives thereof.

More examples of detection marker can also be found in U.S. Pat. Nos. 5,723,591 and 5,928,907; WO2011066476 and WO2012149042; www.idahotech.com; Gudnason et al., NucleicAcids Res., 35(19): e127(2007), which is fully incorporated into the invention by reference.

Detection markers can be linked to oligonucleotide molecules by covalent or non-covalent bonds. Noncovalent bonds include, but not limited to, hydrogen bond, ion bond, van der Waals force and hydrophobic bond. For example, in some embodiments, detection marker may be linked to a nucleotide molecule by covalent bond. For example, amino allyl UTP can be incorporated in the synthesis of oligonucleotide molecules, and the generated amino allyl-labeled nucleic acid molecules can be coupled to fluorophores containing NHS-ester (such as Alexa 488, Alexa 594, Alexa 647 (Invitrogen) or Cy3 (GE Healthcare)) to form covalent bond link.

In some embodiments, the detection marker is selected from: FAM, Tetrachlorofluorescein (TET), Alexa 488, Alexa 532, CF, HEX, VIC, ROX, Texas Red, QuasarFITC, cy3, cy5, 6-joe, EDANS, rhodamine 6G (P6G), tetramethyirhodamine (TMR), tetramethylrhodamine isothiocyanate (TMRITC), x-rhodamine, Texas red, biotin and avidin.

In some embodiments, the probe contains a quenchable signal. “quencher” in the invention refers to a molecule capable of preventing detection marker from generating detection signal, wherever spatially close enough to the detection marker. A quencher cannot prevent generation of detection signal when the quencher is far away from the detection marker.

Examples of quenching molecules include, but not limited to, DDQ-I, DDQ-II, Dabcyl, Eclipse, Iowa Black FQ, Iowa Black RQ, BHQ-1, BHQ-2, BHQ-3, “QSY7”, “QSY-21” and “QSY33” (Molecular Probe), Ferrocene and the derivative thereof, methyl viologen, tetramethylrhodamine (TAMRA), Minor groove binding non-fluorescent quencher (MGBNFQ) and N,N′-dimethyl-2,9-diazopyrenium.

In some embodiments, the fluorophore is FAM, the quenching molecule is MGBNFQ or DDQ-I. In some embodiments, the fluorophore is TAMRA, Cy3, ROX, Cy5, the quenching molecule is DDQ-II. In some embodiments, the fluorophore is FAM, HEX, ROX, JOE, the quenching molecule is Dabcyl. In some embodiments, the probe has the fluorophore FAM or VIC at the 5′ end and the quenching molecule MGBNFQ at the 3′ end.

A quenching molecule can be linked to the probe by a method well known in the art. For example, amino-allyl UTP can be incorporated in the synthesis of oligonucleotide molecules, and the generated amino allyl-labeled nucleic acid molecules can be coupled to quenching molecules containing NHS-ester to form covalent bond link. For another example, a quenching molecule can be linked to oligonucleotide at the 3′ end through reacting with phosphorous amide derivatives of the quenching molecule (such as Dabcyl) during oligonucleotide synthesis process.

In some embodiments, the signal is quenched when the probe is intact. In some embodiments, a detection marker and a quencher are linked to the 5′ end and 3′ end of a probe respectively. For example, for a non-mutation region-probe, a detection marker is linked to the 5′ end, and a quencher is linked to the 3′ end, or a detection marker linked to the 3′ end, and a quencher linked to the 5′ end.

In some embodiments, a polymerase with 5′-3′ exonuclease activity is used to amplify a fragment containing SEG ID No: 1 or the complementary sequence thereof, or use the SEQ ID No: 1 sequence or a fragment of the complementary sequence thereof as the template sequence, and add the probe into the reaction mixture. During the amplification process, the probe will be degraded by polymerase during polymerization reaction when the probe hybridizes with template sequence, thereby the fluorophore on the probe is separated from the quenching molecule and a fluorescent signal is generated (see U.S. Pat. Nos. 5,210,015 and 5,487,972).

The term “fragment” herein refers to a sequence that lacks partial nucleotide sequence at the 5′ end and/or 3′ end, compared to the sequence shown in SEQ ID NO:1 or the reverse 100% complimentary fragment thereof, for example, a sequence lacks 1-70, 2-70, 5-70, 10-70, 20-70, 30-70, 40-70, 50-70, 60-70 nucleotides at the 5′ end, or a sequence lacks 1-70, 2-70, 5-70, 10-70, 20-70, 30-70, 40-70, 50-70, 60-70 nucleotides at the 3′ end, or a sequence lacks 1-70, 2-70, 5-70, 10-70, 20-70, 30-70, 40-70, 50-70, 60-70 nucleotides at both the 5′ end and 3′ end simultaneously, compared to SEQ ID NO:1. According to the understanding of those skilled in the art, the fragment can still amplify the same or similar length as in the embodiments of the invention after PCR amplification, and also achieve the purpose of detecting and distinguishing between human DNA and non-human tissue DNA. It is to be understood by those of skilled in the art that, as for the PCR for SEQ ID NO:1, the length of the final PCR product fragment obtained by primer and probe can be the full length of SEQ ID NO:1, or a part thereof.

### EXAMPLES

The invention will be further illustrated below with reference to the specific examples. These examples are only used to describe the invention but should never be interpreted as to limit in any way the scope or contents of the invention.

### Example 1: Verification of Specificity of the Detection Method

1. Primer Design and Synthesis

The invention designs multiple pairs of primers and probes based on human specific DNA sequence Seq1(SEQ ID NO:1), and meanwhile found from literature and patents three groups of human specific gene and the primers thereof (SRGAP2, Qhomo2 and Alu) to be the control, wherein the primers for Qhomo2 were quoted from the literature (“Preclinical safety study of umbilical cord mesenchymal stem cells”, Youwei Wang, Peking Union Medical College, 2013), according to which the primers can detect human DNA specifically. The primers for SRGAP2 were quoted from the patent CN201910477468.2 “primers for specific detection of human genomic DNA and the use thereof”, according to which the primers could detect human specific DNA sequences from the DNA of many species (including cynomolgus monkeys, rats, mice and New Zealand rabbits). The gene Alu sequence amplified by primer Alu is a universal, diverse and specific short repetitive sequence in human genome. Alu family elements can be used for individual identification in forensic DNA analysis. It is also reported in the literature that Alu sequence can be used to distinguish human DNA from other species. The Alu primers and its probe of the invention were quoted from the literature. Table 1.1 shows the sequences of all primers and probes used in the invention, wherein the 5′ end of the probe contains a reporter group and the 3′ end contains a quencher group.

TABLE 1.1
Primer sequence list for human specific DNA detection

| | | | SEQ |
|---|---|---|---|
| Primer Name | | Sequence (5′ to 3′) | ID NO |
| Seq1 | Primer R1 | ggggaagcatgtogtttcta | 2 |
| | Primer F1 | tcttggatcagggaggagaa | 3 |
| | Primer R2 | acctccctatcacctccgat | 4 |
| | Primer F2 | cttcctacagacgctcccat | 5 |
| | Primer R3 | gggttagtattgggggaagc | 6 |
| | Primer F3 | atgaagctgcctctcttgga | 7 |
| | Primer R4 | ggatgggaaaggaaaatgaag | 8 |
| | Primer F4 | ttcctacagacgctcccatt | 9 |
| | Probe Probe1 | cacttcattttcctttcccatcctt | 10 |
| | Probe Probe2 | cctccctgatccaagagaggcagc | 11 |
| SRGAP2 | Primer SRGAP2-F | cgatactcaggtcaaaggtaagg | 12 |
| | Primer SRGAP2-R | ctgcaaatcacggtggaaatac | 13 |
| | Probe SRGAP2-Probe | tgcaaatgctctgtggactggtga | 14 |
| Qhomo2 | Primer Qhomo2-F | gtgggtgggaagagggaagc | 15 |
| | Primer Qhomo2-R | actcggcattcacacatttctcag | 16 |
| | Probe Qhomo2-Probe | cagcagtggcgtgtgggaacctg | 17 |
| Alu | Primer Alu-F | gtcaggagatcgagaccatcct | 18 |
| | Primer Alu-R | agtggcgcaatctcggc | 19 |
| | Probe Alu-Probe | agctactcgggaggctgaggcagga | 20 |

2. DNA Extraction

Use tissue and cell DNA extraction kit to extract genomic DNA derived from cell and tissue of different species (human, rhesus monkey, green monkey, cynomolgus monkey, rat, mouse and rabbit) according to the instruction of the kit.

3. Taqman PCR Procedure

(1) Reaction system of Taqman qPCR amplification, 20 μL per sample: 10 μL 2×SuperReal PreMix (Probe); 1 μL 50×ROX Reference Dye; 0.6 μL Primer R (10 μM); 0.6 μL Primer F (10 μM); 0.4 μL Probe (10 μM); add in 20 ng of DNA; make up the reaction system to 20 μL with RNase-Free ddH₂O. The primers and probes and combinations thereof used in the procedure are shown in the table below, with totally 13 combination groups:

TABLE 1.2
Primer combination list for human specific DNA detection

| | |
|---|---|
| Target | Combination of primer and probe |

| Number | fragment | R | F | Probe |
|---|---|---|---|---|
| 1. | Seq1 | R1 | F1 | Probe1 |
| 2. | | R1 | F2 | Probe 1 |
| 3. | | R2 | F1 | Probe 1 |
| 4. | | R3 | F3 | Probe 1 |
| 5. | | R2 | F2 | Probe 2 |
| 6. | | R2 | F2 | Probe 1 |
| 7. | | R1 | F2 | Probe 2 |
| 8. | | R4 | F4 | Probe 2 |
| 9. | | R2 | F3 | Probe 1 |
| 10. | | R3 | R4 | Probe 2 |
| 11. | SRGAP2 | R | F | SRGAP2-Probe |
| 12. | Qhomo2 | R | F | Homo4-Probe |
| 13. | Alu | R | F | Alu-Probe |

(2) Reaction condition for Taqman qPCR amplification: Pre-denaturation at 95° C. for 15 minutes; Denaturation at 95° C. for 1 second; Annealing and extension at 62° C. for 30 seconds, with 40 cycles in total; collecting fluorescence signals at 62° C. After the experiment, the CT value was obtained from the instrument.
(3) According to the CT values obtained from experiment in (2), determining the effect of the primers and probes in detecting human DNA: a CT value indicates amplification, and no CT value indicates no amplification. If a combination of primers and probes has amplification in human DNA sample only but not in other species, it indicates that the combination can amplify human specific DNA sequence.

4. Experimental Results

TABLE 1.3
Statistical table of human specific DNA detection results

| | Combination of | | | | | | | | | |
|---|---|---|---|---|---|---|---|---|---|---|
| | primer and | | | Origin of DNA (concentration: 20 ng) | | | | | | |
| Target | probe | | | | rhesus | green | cynomolgus | | | |
| fragment | R | F | Probe | Human | monkey | monkey | monkey | rat | mouse | rabbit |
| Seq1 | R1 | F1 | Probe 1 | + | − | − | − | − | − | − |
| | R1 | F2 | Probe 1 | + | − | − | − | − | − | − |
| | R2 | Fl | Probe 1 | + | − | − | − | − | − | − |
| | R3 | F3 | Probe 1 | + | − | − | − | − | − | − |
| | R2 | F2 | Probe 2 | + | + | / | / | / | / | / |
| | R2 | F2 | Probe 1 | + | + | / | / | / | / | / |
| | R1 | F2 | Probe 2 | + | + | / | / | / | / | / |
| | R4 | F4 | Probe 2 | + | + | / | / | / | / | / |
| | R2 | F3 | Probe 1 | + | − | − | − | − | − | − |
| | R3 | R4 | Probe 2 | + | + | − | − | − | − | − |
| SRGAP2 | R | F | SRGAP2- | + | + | + | + | − | − | − |
| | | | Probe | | | | | | | |
| Qhomo2 | R | F | Homo4- | + | + | − | + | − | − | + |
| | | | Probe | | | | | | | |
| Alu | R | F | Alu- | + | + | + | + | + | + | + |
| | | | Probe | | | | | | | |

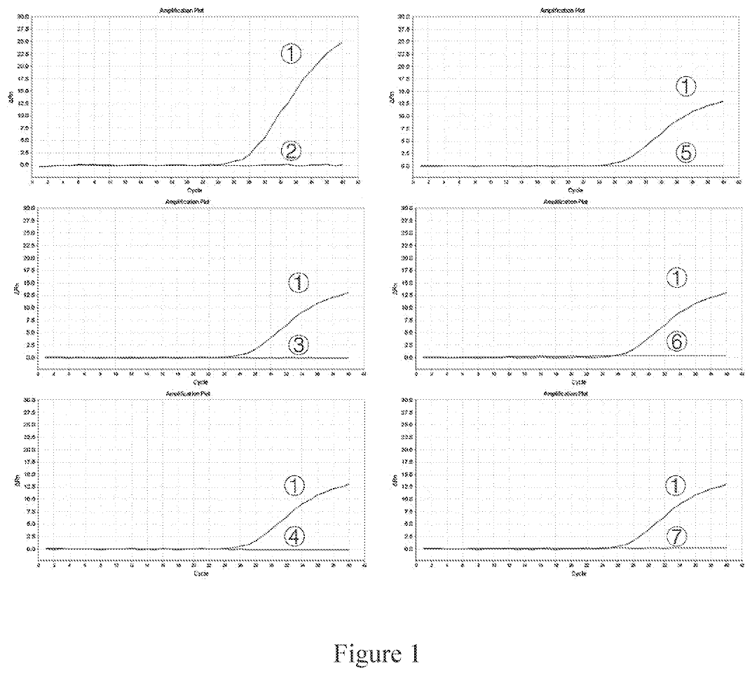

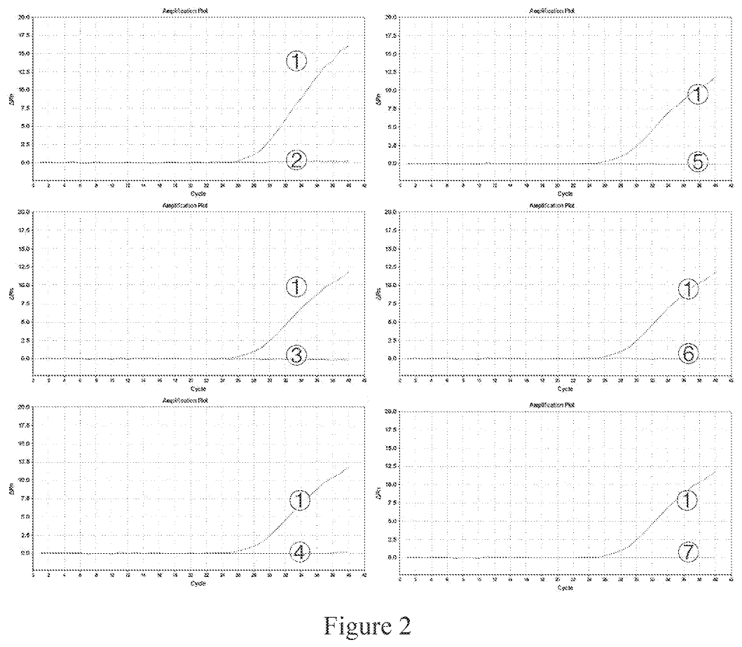

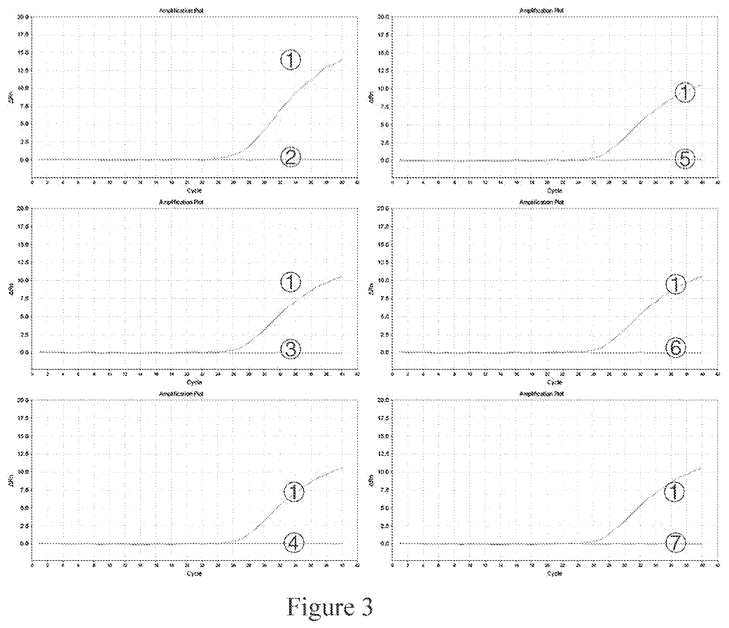

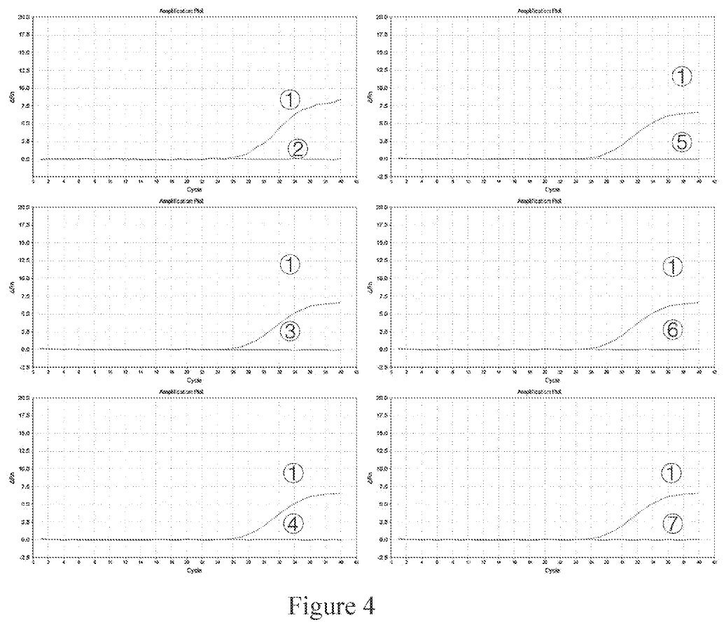

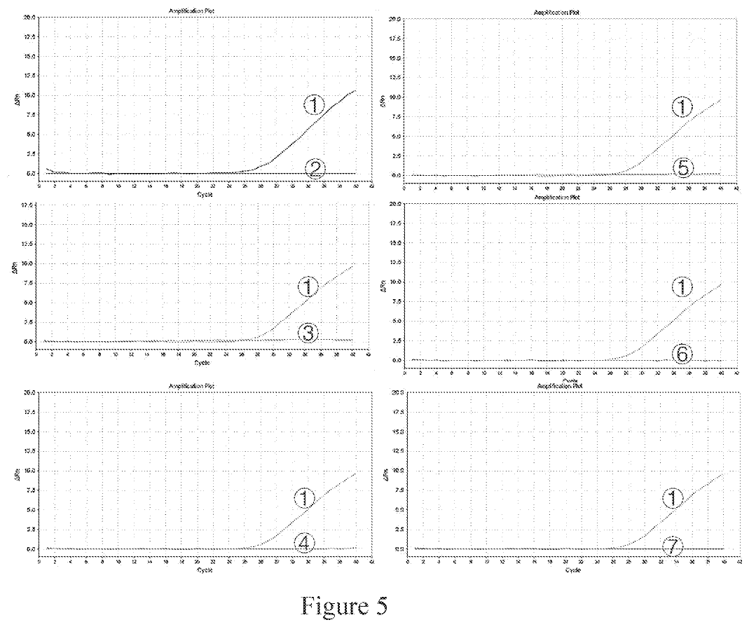

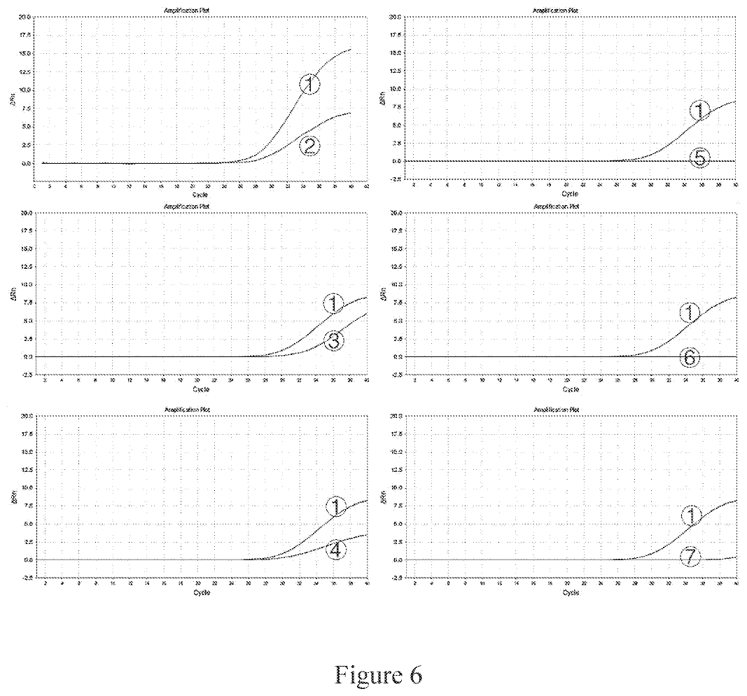

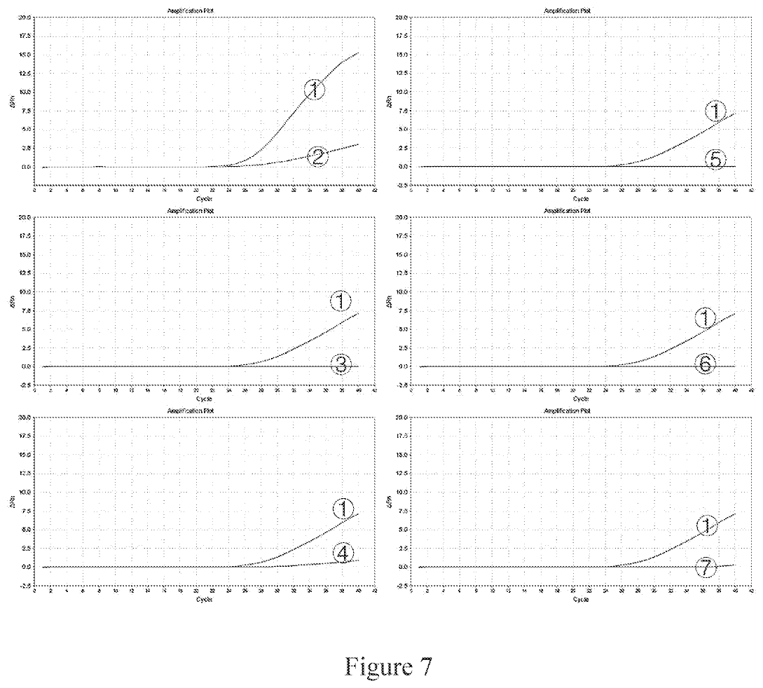

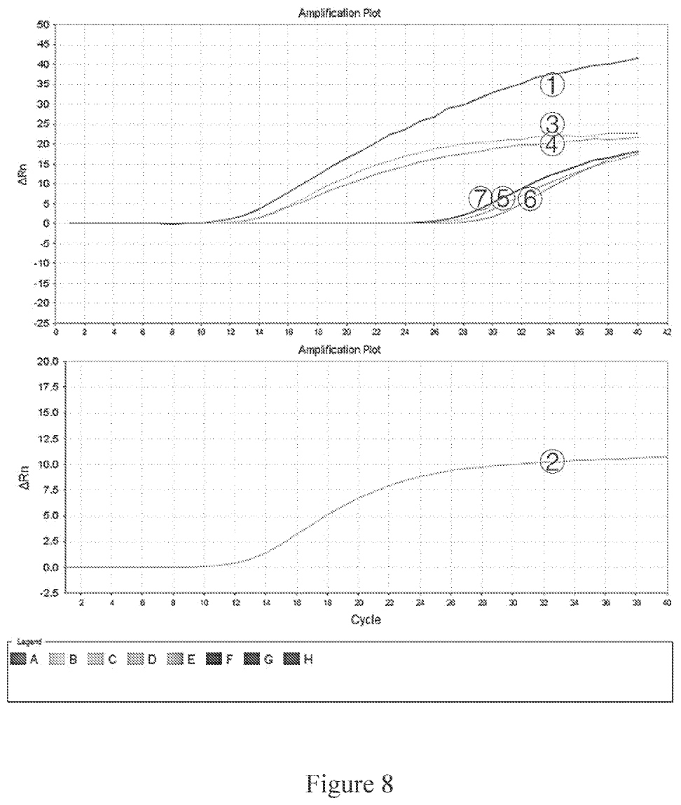

Table 1.3 shows the experimental results, wherein “+” indicates amplification, “−” indicates no amplification; “/” indicates no experiment performed. SRGAP2, Qhomo2 and Alu are human specific genes which have been reported in literature and patents, and the probes and primers are both from reports. In fact, verification in our experiment shows that only Qhomo2 can distinguish between human and green monkey DNA in experiment of distinguishing between human and experimental monkey DNA, and neither SRGAP2 nor Alu can distinguish between human and three experimental monkey (green monkey, cynomolgus monkey and rhesus monkey) DNA. For the DNA sequence Seq1 discovered in the invention, some combination of primers and probes designed for the DNA sequence (such as R2, F2 and Probe2) cannot distinguish between human and monkey DNA, and some (such as R1, F1 and Probe1) can distinguish DNA between human and multiple species (including three kinds of monkeys, rats, mice and rabbits). FIG. 1, FIG. 6, FIG. 7 and FIG. 8 respectively are the amplification curves by primer combination R1 and F1 plus Probe1, SRGAP2, Qhomo2 and Alu. FIGS. 2-5 show amplification curves of DNA sequence Seq1 amplified by other primer combinations. The experimental results also indicate that the primers and probes synthesized in the invention can detect human DNA in multiple species.

TABLE 1.4
Primer position

| | | | Primer | Primer | |
|---|---|---|---|---|---|
| | | | starting | ending | Primer |
| | | | position | position | length |
| Primer name | | Sequence (5′ to 3′) | (5′ end) | (3′ end) | (bp) |
| SEQ ID | Primer R1 | ggggaagcatgtcgtttcta | 71 | 90 | 20 |
| NO: 1 | | | | | |
| | Primer F1 | tcttggatcagggaggagaa | 135 | 116 | 20 |
| | Primer R2 | acctccctatcacctccgat | 8 | 27 | 20 |
| | Primer F2 | cttcctacagacgctcccat | 185 | 166 | 20 |
| | Primer R3 | gggttagtattgggggaagc | 59 | 78 | 20 |
| | Primer F3 | atgaagctgcctctcttgga | 148 | 129 | 20 |
| | Primer R4 | ggatgggaaaggaaaatgaag | 149 | 129 | 21 |
| | Primer F4 | ttcctacagacgctcccatt | 92 | 112 | 20 |
| | Probe Probe1 | cacttcattttcctttcccatcctt | 114 | 90 | 25 |
| | Probe Probe2 | cctccctgatccaagagaggcagc | 120 | 143 | 24 |

TABLE 1.5
Amplified fragment length

| Target | Primer and probe combination | | | Amplified fragment |
|---|---|---|---|---|
| fragment | R | F | Probe | length (bp) |
| SEQ ID NO: 1 | R1 | F1 | Probe1 | 65 |
| | R1 | F2 | Probe 1 | 115 |
| | R2 | F1 | Probe 1 | 128 |
| | R3 | F3 | Probe 1 | 90 |
| | R2 | F2 | Probe 2 | 178 |
| | R2 | F2 | Probe 1 | 178 |
| | R1 | F2 | Probe 2 | 115 |
| | R4 | F4 | Probe 2 | 93 |
| | R2 | F3 | Probe 1 | 141 |
| | R3 | R4 | Probe 2 | 126 |

### Example 2: Quantitative Method Development and Methodological Validation

5 groups of primers and probes combination that can distinguish between human DNA and three types of monkey DNA: R1 (SEQ ID NO:2), F1 (SEQ ID NO:3) and Probe1 (SEQ ID NO:10); R1 (SEQ ID NO:2), F2 (SEQ ID NO:5) and Probe1 (SEQ ID NO:10); R2 (SEQ ID NO:4), F1 (SEQ ID NO:3) and Probe1 (SEQ ID NO:10); R3 (SEQ ID NO:6), F3 (SEQ ID NO:7) and Probe1 (SEQ ID NO:10); R2 (SEQ ID NO:4), F3 (SEQ ID NO:7) and Probe1 (SEQ ID NO:10). Only the combination of one pair of primers and probe (R1, F1 and Probe1) is taken as an example to develop the quantitative method and verify the methodology below. It should be understood by those skilled in the art that other primers have good specificity due to the ability to distinguish between humans and other species, and therefore should have similar methodological validation effects.

### 1. PREPARATION OF STANDARD CURVE AND QUALITY CONTROL SAMPLES

1.1 Preparation of Standard Curve Samples

Prepare the standard curve samples according to the table below, wherein the standard sample is whole genome DNA (concentration: about 140 ng/μL) of human retinal pigment epithelial cell injection (a cell solution containing human retinal pigment epithelial cell, which can be used for binocular subretinal injection of rhesus monkeys). Dilute according to the table below. First add a certain volume of pure water, then add the corresponding volume of whole genome DNA of human retinal pigment epithelial cell injection and STD1˜STD6 into the centrifuge tube, vortex mix for use.

TABLE 2.1
Preparation of standard curve samples

| | | | Pure | Target |
|---|---|---|---|---|
| Solution | | Extraction | water | concentration |
| Name | Extracted solution | (μL) | (μL) | (ng/μL) |
| STD1 | Whole genome DNA of | 20 | 8 | 100 |
| | human retinal pigment | | | |
| | epithelial cell injection | | | |
| STD2 | STD1 | 20 | 80 | 20 |
| STD3 | STD2 | 20 | 80 | 4 |
| STD4 | STD3 | 20 | 80 | 0.8 |
| STD5 | STD4 | 20 | 80 | 0.16 |
| STD6 | STD5 | 20 | 80 | 0.032 |

Note:
the above preparations can be scaled up or down according to actual needs.

1.2 Preparation of Quality Control Sample

Prepare quality control samples according to the table below, wherein the standard sample is whole genome DNA of human retinal pigment epithelial cells (concentration: about 140 ng/μL). Dilute according to the table blow. First add a certain volume of pure water, and add corresponding volume of whole genome DNA of human retinal pigment epithelial cells, upper limit of quantification ULOQ, high quality control HQC, moderate quality control MQC, quality control C, low quality control LQC and lower limit of quantification LLOQ to prepare ULOQ (100 ng/μL), HQC (80 ng/μL), MQC (3.2 ng/μL), C (0.8 ng/μL), LQC (0.08 ng/μL) and LLOQ (0.032 ng/μL).

TABLE 2.2
Preparation of quality control samples

| | | | Pure | Target |
|---|---|---|---|---|
| Solution | | Extraction | water | concentration |
| name | Extracted solution | (μL) | (μL) | (ng/μL) |
| ULOQ | Whole genome DNA of | 20 | 8 | 100 |
| | human retinal pigment | | | |
| | epithelial cell | | | |
| HQC | ULOQ | 20 | 5 | 80 |
| MQC | HQC | 10 | 240 | 3.2 |
| C | MQC | 10 | 30 | 0.8 |
| LQC | C | 10 | 90 | 0.08 |
| LLOQ | LQC | 10 | 15 | 0.032 |

Note:
the above preparations can be scaled up or down according to actual needs.

### 2. DETECTION PROCEDURE

2.1 DNA Extraction

Using tissue and cell DNA extraction kit, extract genomic DNA of cell and tissue according to instructions of the kit.

2.2 Taqman qPCR Procedure

(1) The reaction system of Taqman qPCR amplification for each sample was 20 μL: 10 μL 2×SuperReal PreMix (Probe); 1 μL 50×ROX Reference Dye; 0.6 μL primer R (10 μM); 0.6 μL primer F (10 IM); 0.4 μL probe Probe (10 μM); add in 2 μL of DNA (the DNA templates were whole genome DNA of human retinal pigment epithelial cell, standard curve sample, quality control sample, sample for test, blank matrix negative control sample (Neg) and pure water no template negative control (NTC)); make up the reaction system to 20 μL with RNase-Free ddH₂O.
(2) Reaction condition for Taqman qPCR amplification: Pre-denaturation at 95° C. for 15 minutes; Denaturation at 95° C. for 1 second; Annealing and extension at 62° C. for 30 seconds, with 40 cycles in total; collecting fluorescence signals at 62° C. After the experiment, the CT value, amplification efficiency (Efficiency), R² of standard curve and slope and intercept of standard curve equation were obtained from the instrument.

### 3. DATA PROCESSING

Calculate DNA concentrations of standard curve samples, HQC, MQC and LQC samples and target fragment of sample for test, etc., from CT value, amplification efficiency (Efficiency), R² and the slope and intercept of standard curve equation obtained from standard sample. Conc.=10^{(CT value-y-int)/Slope}, the concentration data shall be rounded to three decimal places, and % Re (relative error) and % CV (coefficient of variation) retain 2 decimal places.

The % RE, standard deviation (SD), % CV and target DNA concentration, etc. used in the report were all calculated by Office Excel 2010 (Microsoft Corporation, USA) software. The calculation formula is as follows:
Average value:

$${\overline{C}}_{t} ( Mean ) = \frac{{\sum }_{i = 1}^{n} {C}_{ti}}{n}$$

(Cₜ is measured concentration);
Relative error percentage:

$$\% R E = \frac{{C}_{t} - {C}_{n}}{{C}_{n}} \times 100 \%$$

(Cₜ is measured concentration, Cn is theoretical concentration);
Percentage of coefficient of variation

$$\% C V = \frac{\sqrt{\frac{{\sum }_{i = 1}^{n} {( {C}_{ti} - {\overline{C}}_{t} )}^{2}}{n - 1}}}{{\overline{C}}_{t}} \times 100 \%$$

(C̅ₜ is average value of measured concentration);
Standard deviation:

$$STDEV ( S D ) = \sqrt{\frac{{\sum }_{i = 1}^{n} {( {C}_{ti} - {\overline{C}}_{t} )}^{2}}{n - 1}}$$

(C̅ₜ is average value of measured concentration);
Sample judgment criteria: 1. when the CT values of LLOQ and LOD were less than those of NTC and Neg: (1) the CT values of sample tested in duplicates were all less than the average CT value of LLOQ, and the concentration result was issued; (2) the CT values of sample tested in duplicates were less than the average CT value of LLOQ, indicating that the sample was positive; (3) if the LOD CT had no value, the concentration results for the batch of samples were issued; 2. when one of the CT values of NEG and NTC was less than that of LOD or the LOD CT had no value, the concentration result was issued only when the CT value of sample tested in duplicates was less than the average CT value of LLOQ; the positive samples are those with CT values between the minimum CT value of NTC and CT value of LLOQ; 3. if one of the CT values of NEG and NTC was less than that of LLOD, the concentration result was issued only when the CT values of sample tested in duplicates were all less than the minimum CT value of Neg and NTC.

### 4. METHODOLOGY VALIDATION RESULT

4.1 Standard Curve and Lower Limit of Quantification

Prepare standard curve sample according to experimental scheme 1.1, and obtain the CT value, amplification efficiency (Efficiency), R² and the slope and intercept of standard curve equation after on-board detection, so as to obtain the standard curve. Determine the linear range and lower limit of quantification of the method (the lowest point of the standard curve). At least two people should verify at least six analytical batches in at least two days, and count the relative error (RE %) between each concentration in each analytical batch and the theoretical concentration, as well as the average relative error (RE %) and precision (CV %) of each concentration between all analytical batches.

Acceptance criteria: the sample concentration of standard curve within and between analytical batches meet the relative error (RE %) between −75%˜150%; inter assay precision (CV %)≤60.00%; all analytical batches should meet R²≥0.980.

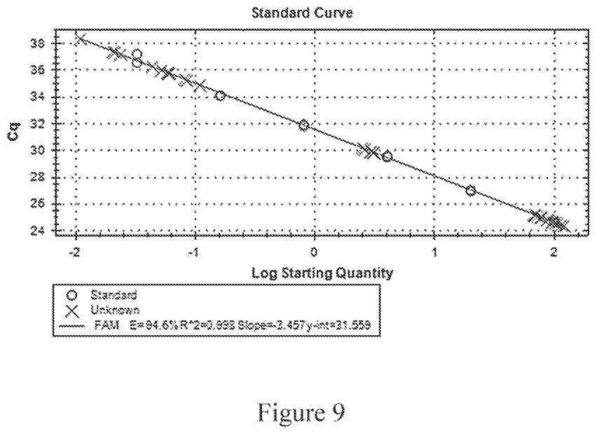

The result showed that: the linear range of the standard curve for qPCR detection of the target fragment of human retinal pigment epithelial cell injection was: 0.032˜100.000 ng/μL, the quantitative lower limit was: 0.032 ng/μL. R² was in the range of 0.991˜0.999; the intra batch accuracy % RE of each concentration point of the standard curve was within the range of −16.75˜43.75%; the inter batch accuracy % RE of each concentration point is within the range of −4.53˜12.50% and the precision % CV of each batch was within the range of 5.42˜13.89%; meeting the accuracy and precision requirements of standard curve. Specific results are shown in Table 2.3, and the typical standard curve is shown in FIG. 9. The summary of standard curve fitting parameters was shown in Table 2.4.

TABLE 2.3
Standard curve results of qPCR for detecting target DNA sequences from human retinal pigment
epithelial cell injection

| Analytical | 0.032 | | 0.160 | | 0.800 | | 4.000 | | 20.000 | | 100.000 | |
|---|---|---|---|---|---|---|---|---|---|---|---|---|
| batch | (ng/μL) | % RE | (ng/μL) | % RE | (ng/μL) | % RE | (ng/μL) | % RE | (ng/μL) | % RE | (ng/μL) | % RE |
| 1 | 0.030 | −6.25 | 0.183 | 14.38 | 0.806 | 0.75 | 3.829 | −4.28 | 20.892 | 4.46 | 96.429 | −3.57 |
| 3 | 0.042 | 31.25 | 0.134 | −16.25 | 0.768 | −4.00 | 3.770 | −5.75 | 21.275 | 6.37 | 104.975 | 4.97 |
| 4 | 0.037 | 15.63 | 0.144 | −10.00 | 0.826 | 3.25 | 3.957 | −1.08 | 19.129 | −4.35 | 106.281 | 6.28 |
| 5 | 0.034 | 6.25 | 0.174 | 8.75 | 0.772 | −3.50 | 3.546 | −11.35 | 18.130 | −9.35 | 115.944 | 15.94 |
| 6 | 0.036 | 12.50 | 0.167 | 4.38 | 0.763 | −4.63 | 3.594 | −10.15 | 19.248 | −3.76 | 111.235 | 11.24 |
| 7 | 0.046 | 43.75 | 0.136 | −15.00 | 0.666 | −16.75 | 3.718 | −7.05 | 20.971 | 4.86 | 113.398 | 13.40 |
| 8 | 0.035 | 9.38 | 0.146 | −8.75 | 0.838 | 4.75 | 4.080 | 2.00 | 19.857 | −0.72 | 100.387 | 0.39 |
| 9 | 0.032 | 0.00 | 0.159 | −0.63 | 0.835 | 4.37 | 3.728 | −6.80 | 22.527 | 12.64 | 93.869 | −6.13 |
| 10 | 0.035 | 9.38 | 0.144 | −10.00 | 0.904 | 13.00 | 4.148 | 3.70 | 20.553 | 2.77 | 93.579 | −6.42 |
| Averag | 0.036 | / | 0.154 | / | 0.798 | / | 3.819 | / | 20.287 | / | 104.011 | / |
| SD | 0.005 | / | 0.017 | / | 0.066 | / | 0.207 | / | 1.33 | / | 8.449 | / |
| % CV | 13.89 | / | 11.04 | / | 8.27 | / | 5.42 | / | 6.56 | / | 8.12 | / |
| % RE | 12.50 | / | −3.75 | / | −0.25 | / | −4.53 | / | 1.44 | / | 4.01 | / |
| n | 9 | / | 9 | / | 9 | / | 9 | / | 9 | / | 9 | / |

Note:
“/” indicates no calculation data.

TABLE 2.4
Summary of standard curve fitting parameters

| Analytical | standard curve parameter | | | |
|---|---|---|---|---|
| batch | Slope | y-int | E (Efficiency) | R² |
| 1 | −3.457 | 31.559 | 94.6 | 0.998 |
| 3 | −3.343 | 31.069 | 99.1 | 0.994 |
| 4 | −3.433 | 31.332 | 95.6 | 0.997 |
| 5 | −3.487 | 31.648 | 93.6 | 0.998 |
| 6 | −3.359 | 32.027 | 98.5 | 0.996 |
| 7 | −3.556 | 31.776 | 91.1 | 0.991 |
| 8 | −3.515 | 31.294 | 92.5 | 0.997 |
| 9 | −3.478 | 31.016 | 93.9 | 0.999 |
| 10 | −3.700 | 31.094 | 86.3 | 0.995 |

Note:
Standard curve fitting formula: CT = Slope LgX₀ + y-int; wherein X₀ is the initial concentration of the samples, y-int is the intercept, Slope is the slope.

4.2 Precision and Accuracy

To verify the intra and inter batch precision (Precision) and relative error (Accuracy) of this method, 3 sets of quality control samples with 5 concentrations of ULOQ, HQC, MQC, LQC, LLOQ in the same analytical batch were prepared according to Table 2.2. At least 2 people should verify at least 6 analytical batches in at least 2 days. The precision (CV %) and average relative error (% RE) within each concentration batch of quality control samples, as well as the total precision (% CV) and average relative error (RE %) between batches were quantified.

Acceptance criteria: the concentrations within and between batches met the average relative error (RE %) between −75/˜150%; concentrations within and between batches met precision (CV %)≤60.00%.

The result showed that: the intra batch accuracy % RE of each concentration of quality control samples was within the range of −22.50˜41.25%; the intra batch precision was within the range of 1.37˜60.00%; the inter batch accuracy % RE of each concentration of quality control samples was within the range of 0.91˜15.60% and the inter batch precision was within the range of 9.28˜35.71%; meeting the intra and inter batch accuracy and precision requirements. The above data show that the accuracy and precision of the analysis method met the requirements. Specific results are shown in Table 2.5.

TABLE 2.5
Precision and accuracy of qPCR in detecting the target DNA sequence of human retinal pigment
epithelial cell injection

| | Name of quality control | LLOQ | LOC | MQC | HQC | ULOQ |
|---|---|---|---|---|---|---|
| Analytical batch | Theoretical concentration (ng/μL) | 0.032 | 0.08 | 3.2 | 80 | 100 |
| 1 | Measured concentration | 0.032 | 0.082 | 2.714 | 78.418 | 105.825 |
| | (ng/μL) | 0.034 | 0.059 | 2.844 | 80.536 | 117.726 |
| | | 0.021 | 0.055 | 3.019 | 69.558 | 97.696 |
| | Average (ng/μL) | 0.029 | 0.065 | 2.859 | 76.171 | 107.082 |
| | SD | 0.007 | 0.015 | 0.153 | 5.824 | 10.074 |
| | % CV | 24.14 | 23.08 | 5.35 | 7.65 | 9.41 |
| | % RE | −9.38 | −18.75 | −10.66 | −4.79 | 7.08 |
| 3 | Measured concentration | 0.020 | 0.091 | 2.103 | 89.75 | 117.408 |
| | (ng/μL) | 0.049 | 0.141 | 2.906 | 87.915 | 108.094 |
| | | 0.042 | 0.107 | 2.43 | 92.257 | 118.22 |
| | Average (ng/μL) | 0.037 | 0.113 | 2.48 | 89.974 | 114.574 |
| | SD | 0.015 | 0.026 | 0.404 | 2.18 | 5.627 |
| | % CV | 40.54 | 23.01 | 16.29 | 2.42 | 4.91 |
| | % RE | 15.63 | 41.25 | −22.5 | 12.47 | 14.57 |
| 4 | Measured concentration | 0.036 | 0.125 | 2.851 | 108.091 | 118.733 |
| | (ng/μL) | 0.041 | 0.054 | 3.417 | 101.759 | 114.817 |
| | | 0.030 | 0.056 | 3.371 | 90.793 | 126.121 |
| | Average (ng/μL) | 0.036 | 0.078 | 3.213 | 100.214 | 119.890 |
| | SD | 0.006 | 0.040 | 0.314 | 8.752 | 5.740 |
| | % CV | 16.67 | 51.28 | 9.77 | 8.73 | 4.79 |
| | % RE | 12.50 | −2.50 | 0.41 | 25.27 | 19.89 |
| 5 | Measured concentration | 0.019 | 0.066 | 2.892 | 84.447 | 112.919 |
| | (ng/μL) | 0.045 | 0.064 | 3.816 | 77.500 | 109.976 |
| | | 0.020 | 0.117 | 3.479 | 81.704 | 112.176 |
| | Average (ng/μL) | 0.028 | 0.082 | 3.396 | 81.217 | 111.690 |
| | SD | 0.015 | 0.03 | 0.468 | 3.499 | 1.53 |
| | % CV | 53.57 | 36.59 | 13.78 | 4.31 | 1.37 |
| | % RE | −12.50 | 2.50 | 6.12 | 1.52 | 11.69 |
| 6 | Measured concentration | 0.034 | 0.079 | 3.499 | 85.942 | 121.909 |
| | (ng/μL) | 0.038 | 0.033 | 3.904 | 104.127 | 144.699 |
| | | 0.021 | 0.129 | 3.825 | 98.571 | 116.997 |
| | Average (ng/μL) | 0.031 | 0.080 | 3.743 | 96.213 | 127.868 |
| | SD | 0.009 | 0.048 | 0.215 | 9.319 | 14.781 |
| | % CV | 29.03 | 60.00 | 5.74 | 9.69 | 11.56 |
| | % RE | −3.13 | 0.00 | 16.97 | 20.27 | 27.87 |
| 7 | Measured concentration | 0.047 | 0.073 | 3.118 | 79.412 | 124.950 |
| | (ng/μL) | 0.019 | 0.073 | 3.572 | 72.530 | 114.122 |
| | | 0.027 | 0.106 | 3.326 | 85.275 | 98.331 |
| | [symbol] (ng/μL) | 0.031 | 0.084 | 3.339 | 79.072 | 112.468 |
| | SD | 0.014 | 0.019 | 0.227 | 6.379 | 13.386 |
| | % CV | 45.16 | 22.62 | 6.80 | 8.07 | 11.90 |
| | % RE | −3.13 | 5.00 | 4.34 | −1.16 | 12.47 |
| Inter batch data calculation | [symbol] (ng/μL) | 0.032 | 0.084 | 3.171 | 87.144 | 115.596 |
| | SD | 0.009 | 0.030 | 0.441 | 10.422 | 10.726 |
| | % CV | 28.13 | 35.71 | 13.91 | 11.96 | 9.28 |
| | % RE | 0.00 | 5.00 | −0.91 | 8.93 | 15.60 |

4.3 Effects of Different Blank Matrix Genome Quality and Concentration on Target

Extract DNA from whole blood, lung, liver, choroid+RPE (retinal pigment epithelial cells) and iris of blank control rhesus monkeys as interference. Add standard curve samples into the reaction system containing 200 ng and 100 ng of blank lung and liver DNA respectively; add standard curve samples into the reaction system containing 100 ng and 40 ng of blank choroid+RPE, iris and whole blood DNA respectively (when the blank matrix DNA was insufficient to make up to the corresponding total amount of DNA, prepare with the actual total amount). Detect the samples, calculate the relative error (RE %) between each concentration of the standard curve with blank DNA and the theoretical concentration.

Acceptance criteria: compare the |RE %| sum of two standard curves adding blank matrix DNA to each tissue, and take the total amount with smaller |RE %| sum as addition amount of template in the reaction system during actual sample detection; if the |RE %| sum of the two sets of standard curves was close, select the total amount in the set of standard curves with smaller |RE %| sum at low concentration point on the standard curve as addition amount of template in the reaction system during actual sample detection, and calculate the optimal detection concentration. When the actual concentration of samples was 20% greater than the optimal detection concentration during actual sample detection, dilute samples to the optimal detection concentration. If the actual sample concentration was less than the optimal detection concentration, carry out the detection according to the actual concentration.

The result showed that, when the total amount of whole blood DNA of blank control rhesus monkeys was 100 ng and 40 ng respectively, the |RE %| sum of two standard curve was 207.11 and 125.32 respectively, and it worked out that the amount of template added in the reaction system was 40 ng, and the optimal detection concentration of samples was 20 ng/μL, when detecting the whole blood sample; when the total amount of lung DNA of blank control rhesus monkeys was 200 ng and 100 ng respectively, the |RE %| sum of two standard curve was 549.81 and 311.49 respectively, and it worked out that the amount of template added in the reaction system was 100 ng, and the optimal detection concentration of samples was 50 ng/μL, when detecting the lung sample; when the total amount of liver DNA of blank control rhesus monkeys was 200 ng and 100 ng respectively, the |RE %| sum of two standard curve was 98.51 and 113.79 respectively, and it worked out that the amount of template added in the reaction system was 200 ng, and the optimal detection concentration of samples was 100 ng/μL, when detecting the liver sample; when the total amount of choroid+RPE DNA of blank control rhesus monkeys was 100 ng and 40 ng respectively, the | RE %| sum of two standard curve was 356.71 and 95.04 respectively, and it worked out that the amount of template added in the reaction system was 40 ng, and the optimal detection concentration of samples was 20 ng/μL, when detecting the choroid+RPE sample; when the total amount of iris DNA of blank control rhesus monkeys was 100 ng and 40 ng respectively, the |RE %| sum of two standard curve was 576.19 and 218.31 respectively, and it worked out that the amount of template added in the reaction system was 40 ng, and the optimal detection concentration of samples was 20 ng/μL, when detecting the iris sample. Specific results are shown in Table 2.6.

TABLE 2.6
Effects of different blank matrix genome quality and concentration
of rhesus monkey on target DNA sequence detection

| Blank | Analytical | | Real | Theoretical | |
|---|---|---|---|---|---|
| matrix | batch | Sample | Conc. Mean | Conc. | % RE |
| Whole blood | 9 | STD1-Blood100 ng | 77.333 | 100 | 22.67 |
| DNA | | STD2-Blood100 ng | 13.665 | 20 | −31.68 |
| | | STD3-Blood100 ng | 2.755 | 4 | −31.13 |
| | | STD4-Blood100 ng | 0.452 | 0.8 | −43.5 |
| | | STD5-Blood100 ng | 0.085 | 0.16 | −46.88 |
| | | STD6-Blood100 ng | 0.022 | 0.032 | −31.25 |
| | | Sum \| % RE \| | / | / | 207.11 |
| | 8 | STD1-Blood40 ng | 132.944 | 100 | 32.94 |
| | | STD2-Blood40 ng | 20.714 | 20 | 3.57 |
| | | STD3-Blood40 ng | 4.067 | 4 | 1.68 |
| | | STD4-Blood40 ng | 0.802 | 0.8 | 0.25 |
| | | STD5-Blood40 ng | 0.179 | 0.16 | 11.88 |
| | | STD6-Blood40 ng | 0.056 | 0.032 | 75.00 |
| | | Sum \| % RE \| | / | / | 125.32 |
| Liver DNA | 9 | STD1-Liver200 ng | 123.579 | 100 | 23.58 |
| | | STD2-Liver200 ng | 17.191 | 20 | −14.05 |
| | | STD3-Liver200 ng | 3.52 | 4 | −12.00 |
| | | STD4-Liver200 ng | 0.709 | 0.8 | −11.38 |
| | | STD5-Liver200 ng | 0.12 | 0.16 | −25.00 |
| | | STD6-Liver200 ng | 0.028 | 0.032 | −12.50 |
| | | Sum \| % RE \| | / | / | 98.51 |
| | 9 | STD1-Liver100 ng | 124.638 | 100 | 24.64 |
| | | STD2-Liver100 ng | 23.168 | 20 | 15.84 |
| | | STD3-Liver100 ng | 3.683 | 4 | −7.93 |
| | | STD4-Liver100 ng | 0.657 | 0.8 | −17.88 |
| | | STD5-Liver100 ng | 0.166 | 0.16 | 3.75 |
| | | STD6-Liver100 ng | 0.018 | 0.032 | −43.75 |
| | | Sum \| % RE \| | / | / | 113.79 |
| choroid + RPE DNA | 8 | STD1-ChoriodRPE100 ng | 80.754 | 100 | −19.25 |
| | | STD2-ChoriodRPE100 ng | 8.316 | 20 | −58.42 |
| | | STD3-ChoriodRPE100 ng | 1.284 | 4 | −67.90 |
| | | STD4-ChoriodRPE100 ng | 0.181 | 0.8 | −77.38 |
| | | STD5-ChoriodRPE100 ng | 0.071 | 0.16 | −55.63 |
| | | STD6-ChoriodRPE100 ng | 0.007 | 0.03 | −78.13 |
| | | Sum \| % RE \| | / | / | 356.71 |
| | 8 | STD1-Choriod + RPE40 ng | 115.398 | 100 | 15.40 |
| | | STD2-Choriod + RPE40 ng | 20.077 | 20 | 0.39 |
| | | STD3-Choriod + RPE40 ng | 4.095 | 4 | 2.37 |
| | | STD4-Choriod + RPE40 ng | 0.75 | 0.8 | −6.25 |
| | | STD5-Choriod + RPE40 ng | 0.147 | 0.16 | −8.13 |
| | | STD6-Choriod + RPE40 ng | 0.012 | 0.032 | −62.50 |
| | | Sum \| % RE \| | / | / | 95.04 |
| Iris DNA | 8 | STD1-Iris100 ng | 7.779 | 100 | −92.22 |
| | | STD2-Iris100 ng | 0.562 | 20 | −97.19 |
| | | STD3-Iris100 ng | 0.144 | 4 | −96.40 |
| | | STD4-Iris100 ng | 0.052 | 0.8 | −93.50 |
| | | STD5-Iris100 ng | 0.005 | 0.16 | −96.88 |
| | | STD6-Iris100 ng | 0* | 0.032 | −100.00 |
| | | Sum \| % RE \| | / | / | 576.19 |
| | 8 | STD1-Iris40 ng | 151.98 | 100 | 51.98 |
| | | STD2-Iris40 ng | 24.258 | 20 | 21.29 |
| | | STD3-Iris40 ng | 4.686 | 4 | 17.15 |
| | | STD4-Iris40 ng | 0.787 | 0.8 | −1.63 |
| | | STD5-Iris40 ng | 0.133 | 0.16 | −16.88 |
| | | STD6-Iris40 ng | 0.067 | 0.032 | 109.38 |
| | | Sum \| % RE \| | / | / | 218.31 |
| Lung DNA | 9 | STD1-Lung200 ng | 23.972 | 100 | −76.03 |
| | | STD2-Lung200 ng | 1.737 | 20 | −91.32 |
| | | STD3-Lung200 ng | 0.257 | 4 | −93.58 |
| | | STD4-Lung200 ng | 0.039 | 0.8 | −95.13 |
| | | STD5-Lung200 ng | 0.01 | 0.16 | −93.75 |
| | | STD6-Lung200 ng | 0* | 0.032 | −100.00 |
| | | Sum \| % RE \| | / | / | 549.81 |
| | 9 | STD1-Lung100 ng | 89.59 | 100 | −10.41 |
| | | STD2-Lung100 ng | 11.153 | 20 | −44.24 |
| | | STD3-Lung100 ng | 1.657 | 4 | −58.58 |
| | | STD4-Lung100 ng | 0.264 | 0.8 | −67.00 |
| | | STD5-Lung100 ng | 0.055 | 0.16 | −65.63 |
| | | STD6-Lung100 ng | 0.011 | 0.032 | −65.63 |
| | | Sum \| % RE \| | / | / | 311.49 |

Note:
*indicates no value, the actual concentration was calculated as 0.

4.4 Limit of Detection (LOD)

Dilute LLOQ with pure water into samples with concentrations of S1 (0.016 ng/μL), S2 (0.008 ng/μL), S3 (0.004 ng/μL) and S4 (0.002 ng/μL), 16 single wells for each concentration were tested to determine the sensitivity of the method. Meanwhile, the mixed DNA of whole blood, lung, liver, choroid+RPE and iris of blank control rhesus monkey was used as negative control to determine the detection limit (LOD) of the method. If the sample of a certain concentration had failed to meet the acceptance criteria, the samples with lower concentrations should not be tested.

Acceptance criteria: the limit of detection of this method met the CT value of 60% samples <the CT value of blank mixed DNA or was the lowest concentration that the CT of sensitivity sample had a value but CT of blank mixed DNA had no value. In actual detection, this concentration was used as the limit of detection (LOD). If the CT value of sample for test was greater than the CT value of the lower limit of quantification (LLOQ) and the calculated concentration was greater than the limit of detection (LOD), the sample was defined as positive, but with no exact concentration.

The result showed that, when concentration was S1 (0.016 ng/μL), 62.50% of S1 samples had CT values, and blank mixed DNAs had no value; when concentration was S2 (0.008 ng/μL), CT values of 31.25% of S2 samples <CT values of black mixed DNAs. It is worked out that the limit of detection of the method was S1 (0.016 ng/μL). Specific results are shown in Table 2.7 and Table 2.8.

TABLE 2.7
Limit of detection of qPCR in detecting the target DNA sequence
of human retinal pigment epithelial cell injection (I)

| Analytical | | | |
|---|---|---|---|
| batch | Sample | CT | CT (Neg Ctrl) |
| 6 | S1 | NaN | NaN |
| | S1 | NaN | NaN |
| | S1 | 37.46 | NaN |
| | S1 | 38.16 | NaN |
| | S1 | 37.02 | NaN |
| | S1 | NaN | NaN |
| | S1 | 37.46 | NaN |
| | S1 | 38.47 | NaN |
| | S1 | 35.91 | NaN |
| | S1 | NaN | NaN |
| | S1 | 37.24 | NaN |
| | S1 | 37.16 | NaN |
| | S1 | 38.27 | NaN |
| | S1 | 36.98 | NaN |
| | S1 | NaN | NaN |
| | S1 | NaN | NaN |

Note:
“NaN” indicates no CT value.

TABLE 2.8
Limit of detection of qPCR in detecting the target DNA sequence
of human retinal pigment epithelial cell injection (II)

| Analytical | | | |
|---|---|---|---|
| batch | Sample | CT | CT (Neg Ctrl) |
| 6 | S2 | NaN | NaN |
| | S2 | NaN | NaN |
| | S2 | NaN | NaN |
| | S2 | 38.10 | NaN |
| | S2 | NaN | NaN |
| | S2 | NaN | NaN |
| | S2 | NaN | NaN |
| | S2 | 39.15 | NaN |
| | S2 | NaN | NaN |
| | S2 | 37.07 | NaN |
| | S2 | NaN | NaN |
| | S2 | 37.26 | NaN |
| | S2 | 38.12 | NaN |
| | S2 | NaN | NaN |
| | S2 | NaN | NaN |
| | S2 | NaN | NaN |

Note:
“NaN” indicates no CT value.

4.5 Selectivity

The samples for test in the actual detection include all DNA solutions of each tissue and blood for test. To evaluate the effect of blank matrix (i.e., experimental animal tissue and blood genome) on sample detection, extract DNA from whole blood, lung, liver, choroid+RPE and iris of rhesus monkey and dilute to the optimal detection concentration determined in the above Section “4.3”, detect CT of blank matrix, which required that no obvious endogenous DNA interference affects the positive or negative sample determination.

Acceptance criteria: The CT value of each hole of whole blood, lung, liver, choroid+RPE and iris DNA of blank control rhesus monkey and pure water (NTC) was greater than that of the large CT in LOD duplicates (or the blank matrix showed no CT).

The result showed that, CT values of whole blood, lung, liver, choroid+RPE and iris DNA of blank control rhesus monkey all showed no CT, but only the liver duplicates had one CT of 39.54, which was greater than that of the large CT in LOD duplicates and met the requirement, demonstrating that there was no obvious endogenous DNA interference affecting the positive or negative sample determination. Specific results are shown in Table 2.9.

TABLE 2.9
Selectivity result of qPCR in detecting the target DNA sequence
of human retinal pigment epithelial cell injection

| Analytical | | | large CT value |
|---|---|---|---|
| batch | Sample | CT Mean | in LOD duplicates |
| 10 | Blank-Blood | NaN | 39.03 |
| | Blank-Iris | NaN | 39.03 |
| | Blank-Liver | 39.54 | 39.03 |
| | Blank-Lung | NaN | 39.03 |
| | Blank-Choriod + RPE | NaN | 39.03 |
| | NTC | NaN | 39.03 |

Note:
“NaN” indicates no CT.

### 5. CONCLUSION

The above verification results showed that the linear range of the real-time fluorescent qPCR method for detecting human DNA (human retinal pigment epithelial cell DNA) in rhesus monkeys was: 0.032-100.000 ng/μL, the lower limit of quantification was 0.032 ng/μL, the limit of detection was 0.016 ng/μL; the precision and accuracy met the requirements, with no obvious endogenous DNA interference affecting the positive or negative sample determination, and with fine selectivity, which could be used to detect the concentration of DNA sequence of human retinal pigment epithelial cells in rhesus monkey tissue and blood samples.

The foregoing embodiments are to be considered in all respects illustrative rather than limiting the invention described herein. The scope of the invention is indicated by the appended claims rather than by the foregoing description, and all changes that come within the meaning and the range of equivalents of the claims are intended to be embraced therein.

1. A method for distinguishing between human and non-human animal DNA in a sample mixed with human and non-human animal tissues, comprising
detecting a DNA sequence in a sample mixed with human and non-human animal tissues, wherein the DNA sequence is selected from the group consisting of SEQ ID NO:1, or a fragment thereof, and a reverse complement sequence of SEQ ID NO:1, or a fragment thereof.

2. (canceled)

3. (canceled)

4. The method of claim 3, further comprising amplifying the DNA sequence by PCR technologies technologies using the primers and probes for detecting the DNA sequence.

5. The method of claim 17, wherein sequence of the probe is shown in SEQ ID NO:10.

6. The use of claim 17, wherein the probe is provided with a detection marker, and the detection marker is at least one selected from the group consisting of FAM, TET, Alexa 488, Alexa 532, CF, HEX, VIC, ROX, Texas Red, QuasarFlTC, cy3, cy5, 6-joe, EDANS, rhodamine 6G, TMR, TMRITC, x-rhodamine, Texas red, biotin and avidin.

7. The method use of claim 17, wherein sequence of the primer is selected from the group consisting of SEQ ID NO:2 and SEQ ID NO:3; SEQ ID NO:2 and SEQ ID NO:5; SEQ ID NO:4 and SEQ ID NO:3; SEQ ID NO:6 and SEQ ID NO:7; and SEQ ID NO:4 and SEQ ID NO:7.

8. The method of claim 1, wherein the non-human animal is selected from the group consisting of rhesus monkey, green monkey, cynomolgus monkey, rat, mouse, and rabbit.

9. The method of claim 1, wherein the sample mixed human and non-human animal tissue is tissue or blood sample of rhesus monkey mixed with human DNA, the human DNA being derived from human cells.

10. A composition comprising primers and probes, wherein sequence of the probe is shown in SEQ ID NO:10, sequence of the primer is selected from the group consisting of SEQ ID NO:2 and SEQ ID NO:3; SEQ ID NO:2 and SEQ ID NO:5; SEQ ID NO:4 and SEQ ID NO:3; SEQ ID NO:6 and SEQ ID NO:7; and SEQ ID NO:4 and SEQ ID NO:7.

11. The composition of claim 10, wherein the probe is provided with a detection marker, wherein the detection marker is preferably selected from the group consisting of FAM, TET, Alexa 488, Alexa 532, CF, HEX, VIC, ROX, Texas Red, QuasarFlTC, cy3, cy5, 6-joe, EDANS, rhodamine 6G, TMR, TMRITC, x-rhodamine, Texas red, biotin and avidin.

12. A kit comprising the composition of claim 10.

13. (canceled)

14. (canceled)

15. (canceled)

16. (canceled)

17. The method of claim 1, comprising the steps:
1. performing DNA extraction of a sample mixed with human and non-human animal tissue using cellular DNA extraction kit;
2. performing Taqman qPCR amplification using the primers and probes;
3. collecting a fluorescent signal, calculating a cycle threshold (CT) value, calculating the concentration of human DNA in the sample.

18. The method of claim 9, wherein the human cells are from retinal pigment epithelial cells.
